(19) United States
(12) Patent Application Publication
(10) Pub. No.: US 2013/0236521 A1
(43) Pub. Date: Sep. 12, 2013

(54) SnF2 APPLICATIONS, METHODS AND RELATED FORMULATIONS

(71) Applicant: HERPX LLC, Watchung, NJ (US)

(72) Inventors: Thomas Blake, Budd Lake, NJ (US); Thomas S. Parigian, Ocean, NJ (US); David E. Walsh, Watchung, NJ (US)

(73) Assignee: HERPX LLC, Watchung, NJ (US)

(21) Appl. No.: 13/789,242

(22) Filed: Mar. 7, 2013

Related U.S. Application Data
(60) Provisional application No. 61/608,142, filed on Mar. 8, 2012.

(51) Int. Cl. A61K 33/24; A61K 33/20

(52) U.S. Cl. CPC A61K 33/24; A61K 33/20
USPC 424/402; 424/650; 424/443

(57) ABSTRACT

The present disclosure is directed to method of treating a herpes virus. In one embodiment, the method includes adding an effective amount of stannous fluoride (SnF₂) to a dosage form. The dosage form may be a scrim, a liquid (e.g., a nasal mist, an aerosol spray, a liquid eye drop, a liquid bandage, and the like), a mucoadhesive or a lip balm.

### CROSS REFERENCE TO RELATED APPLICATIONS

This application claims priority under 35 U.S.C. §119(e) to U.S. provisional patent application Ser. No. 61/608,142, filed on Mar. 8, 2012, which is hereby incorporated by reference in its entirety.

### BACKGROUND

Herpes simplex is a viral disease that affects millions of people worldwide. Herpes simplex can be categorized into various different categories. For example oral herpes, also called cold sores or fever blisters, usually occur around the lips. Other types of herpes simplex can include genital herpes, shingles, ocular herpes, and the like. Generally, the herpes simplex produces unsightly sores in the infected area or region. Also, it can be infectious and a public health concern.

Currently, there is no cure for herpes. In addition, there are a limited number of treatment options. Many of the treatment options require a prescription. Alternatively, there are a few over the counter (OTC) options available, but many of the OTC treatments are ineffective or merely palliative. In addition, many of the currently available treatments are only available in topical cream or gel form.

### SUMMARY

In one embodiment, the present disclosure provides method of treating a herpes virus, comprising. In one embodiment, the method comprises providing a scrim comprising a plurality of openings, wherein a size each one of the plurality of openings is a function of a surface tension and a viscosity of stannous fluoride (SnF₂) that is applied, adding an effective amount of SnF₂to the scrim and applying the scrim containing the effective amount of SnF₂ to an area of a body infected with the herpes virus.

In one embodiment, the present disclosure provides a second method of treating a herpes virus. In one embodiment, the method comprises adding an effective amount of SnF₂to a liquid or a semisolid and applying the liquid containing the effective amount of SnF₂ to an area of a body infected with the herpes virus. In one embodiment, semisolid is defined to be in a form that is different from a gel. Rather, the term semisolid may be defined as a substance that has a viscosity and rigidity between a liquid and a solid.

In one embodiment, the present disclosure provides a third method for treating a herpes virus. The method comprises adding an effective amount of SnF₂ to a mucoadhesive and applying the mucoadhesive containing the effective amount of SnF₂ to an area of a body infected with the herpes virus.

In one embodiment, the present disclosure provides a fourth method for treating a herpes virus. The method comprises formulating an effective amount of SnF₂ into a lip balm, forming the lip balm to accommodate a solid lip balm applicator and applying the lip balm containing the effective amount of SnF₂ to an area of a body infected with the herpes virus using the solid lip balm applicator.

### DETAILED DESCRIPTION

The invention will be primarily described within the context of various embodiments for therapeutic delivery and use of stannous fluoride (SnF₂). SnF₂ may be used alone or with other active agents as either a prescription or OTC drug to stop herpes simplex (or other infectious micro-organisms).

In one embodiment, SnF₂ acts as a blocking mechanism or agent which stops the cutaneous herpes virus from adhering to live skin cell (or mucous membrane), which then stops or retards the method of how the herpes virus spreads. In other words, SnF₂ has the ability to fetter natural attachments sites of the herpes virus and prevents the virus from penetrating a host cell. As a result the viral disease process is arrested in the living host.

Normally, the virus migrates to the surface of the skin from subcutaneous nerve cells randomly in individuals with the virus in their system. The virus then attaches to live skin cells (or mucous membrane) on the skin surface and injects its DNA into the live skin cell. The virus then uses the live skin cell as a host to replicate the virus. By stopping the virus adherence to the live skin cell, the virus is stopped from injecting its DNA into the live skin cell and, thus, the virus cannot replicate.

One embodiment of the present disclosure provides a safe and effective prescription or OTC composition of SnF₂, in a gel dosage form. In one embodiment, an example of a manufacturing formula for 0.4% SnF₂ may include 390 pounds (lbs) of glycerin USP, 1.75 lbs of SnF₂ USP, 0.75 lbs of propylparaben NF, 0.25 lbs of methylparaben NF, 1.40 lbs of xylitol NF and 1.50 lbs of rhodigel (xanthan gum USP/NF). Other formulas may be used and the above is provided as only one example. In addition, a variety of standard carriers other than glycerin can be used with SnF₂ or other ingredients.

In addition, the embodiments of the present disclosure provide unique and novel dosage forms for delivering SnF₂, alone or in combination with other active ingredients, to the affected regions or areas. The dosage forms allow for persistent and continuous contact of the SnF₂ to the infected area such that the herpes virus may be effectively blocked. These embodiments include the following:

### Sunscreen or Sunburn Application

Various embodiments contemplate the use of stannous fluoride in combination with sunscreen drug. One embodiment comprises a combination of stannous fluoride gel (e.g., 0.04% SnF2) with any commonly recognized sunscreens, to be formulated into a lotion, aerosol, cream, or pump spray to prevent both sunburn and cold sores in susceptible people. No such combination preparation is known to exist at this time.

### Lip Balm Application

Various embodiments contemplate the use of stannous fluoride in combination with a lip balm, illustrated by such preparations as Chapstick®, Blistex®, Burt's Bees® and the like. In one embodiment, a formulation of 0.4% stannous fluoride in a non-gel form may be combined with a lip balm. In one embodiment, the lip balm may be formed to accommodate a solid lip balm applicator and delivered via the solid and/or semi-solid lip balm applicator or delivery method and/or system that delivers a more liquid stannous fluoride preparation, such as a cream, salve, oil or gel with or without sun block in any color. The lip balm containing the effective amount of the SnF₂ may then be applied to an area of the body (e.g., the lips or the mouth) infected with the herpes virus using the solid or semi-solid lip balm applicator.

Various embodiments contemplate the use of stannous fluoride in various topical dosage forms, such as liquid, gel, cream, hydrocolloid, ointment, oil and powder; molecular forms and isomers such as nano-, stereoisomers, chiral forms, and the like; as well as in concentrations from approximately 0.04% to over 40%. Some embodiments include a sunscreen, while others do not. Some embodiments include the antimicrobial glycerol monolaurate (GML)--in any concentration--while others do not. Some include both sunscreens and GML. Various embodiments comprise should all be in all known forms, liquid, gel, cream, ointment, oil powders, salve, talc, atomized, nano.

### Patch/Scrim Application

Various embodiments contemplate the use of stannous fluoride in various liquid or semisolid forms, delivered via an impregnated patch or scrim. In various embodiments, the physical properties of viscosity and surface tension of the material and/or medium including the stannous fluoride is adapted in viscosity and surface tension to match the characteristics of the patch or scrim, the desired retention level (e.g., retaining on other affected body part). For example, the scrim may be comprised of a plurality of openings. In one embodiment, geometry (e.g. a size of an area of each opening, a width and a length of each opening, and the like) of each one of the openings may be a function of the surface tension and the viscosity of the stannous fluoride, or any liquid or fluid holding the stannous fluoride, to hold the stannous fluoride. Thus, the viscosity and dwell time of the drug in contact with the skin (or mucous membrane) may be adjusted to achieve the desired therapeutic effect. To similar purpose, the margins of the scrim may be crimped or adjusted accordingly.

Therapeutic doses may be delivered via a topical, stannous fluoride gel-impregnated scrim, a scrim impregnated with stannous fluoride gel and another antimicrobial substance, such as GML, chlorine dioxide, or a metallic element (e.g., zinc, silver, tin, or any other metal).

In one embodiment, a unique packaging unit dispenses individualized cutouts of the scrim. In one embodiment a stannous fluoride-containing patch (other than scrim) is provided.

Various embodiments contemplate a topical, stannous fluoride gel-impregnated scrim (mesh) that may holds the drug in place and protects it from being rubbed off. In one variation, the scrim is adapted to offer a sustained-release benefit. In one embodiment, the scrim technology may be capable of incorporating 1) fibers that are clear and small enough not to be noticed, and 2) materials, like those for surgical sutures, that might disappear over time. Optionally, the scrim holds one or more other active ingredients besides the stannous fluoride gel. Individualized cut-outs of the scrim might be dispensed via a unique package, the unit itself possibly amendable to patent protection.

Various embodiments contemplate a stannous fluoride-containing patch. There are other patches besides scrims for holding a drug close to the skin. Some incorporate a drug into the adhesive. Others have drug reservoirs. This dosage form is most appropriate for discrete outbreaks of Herpes zoster (Shingles) and large Herpes simplex infections in areas other than the lips.

### Non-gel Balm Application

Various embodiments contemplate the use of stannous fluoride in liquid or semisolid drug delivery forms other than gels, such as creams, gels, salves, oils, hydrocolloids, lip balm sticks and the like. Also the use in various patches, such as clear, solid, dissolvable and so on. Other contemplated dosage forms include inhalers and eye drops.

### Spray/Pump/Aerosol Application

Various embodiments contemplate the use of stannous fluoride in a spray, pump and/or aerosol form to prevent colds or flu and/or shorten the duration of the cold and/or flu and other upper respiratory infections and/or mitigate or reduces the effect of molds spores and allergens.

### Variuos Concentrations

The various embodiments discussed herein contemplate the use of stannous fluoride in various safe and effective concentrations, such as a 0.4% up to 40%.

### Combination with Cold Sore Preparation

Various embodiments contemplate the use of stannous fluoride in combination with any of the many prescription or OTC cold sore preparations that are local anesthetics, relieve discomfort by making the lips more supple, or that are outright anti-viral, like penciclovir or docosanol.

### Impregnated Particle Application

Various embodiments contemplate the use of stannous fluoride-impregnated articles that bind and capture the smallest of particles (e.g., antigens that may be ionized) that cause airborne allergic reactions. Examples include facemasks, filters on air handling equipment, clothing and the like.

Similarly, since stannous fluoride is a divalent salt, it may be used to bind and capture microorganisms, also ionized that cause airborne infections.

### Impregnated Textile Application

Various embodiments contemplate the use of stannous fluoride-impregnated textiles and clothing that may be used in sterile settings to bind and trap bacteria, viruses, mold spores, allergens and other free-floating microorganisms. Examples include face masks, cloth operating room drapes, sheets/blankets such those used over patients.

### Nasal

Various embodiments contemplate the use of stannous fluoride in an appropriate vehicle, at concentrations known to be safe and effective, as a nasal mist to prevent colds and/or flu (and other upper respiratory infections), or shorten their duration. Various modifications to this embodiment are based upon the indication, the concentration and core formulation, as well as the delivery system. One example is a product designed for airplane travelers, such as those who experience upper respiratory tract infections (URIs) as a result of being in a confined air space. Another example is to prophylactically treat submariners who, after being in a relatively germ-free environment, are known to contract URIs at a frightening rate.

### Anti-Bacterial Embodiments

Various embodiments contemplate the use of stannous fluoride in an appropriate vehicle, at concentrations known to be safe and effective, to combat (prevention and treatment) common, disease-causing bacteria, such as those responsible for such superficial infections (especially those that are nosocomial) as bed sores, pustules, impetigo, infected burns and wounds, etc. A method of delivery is stannous fluoride impregnated wound dressings, such as bandages, gauze, mesh and other such articles.

### Anti-Fungal Embodiments

Various embodiments contemplate the use of stannous fluoride in an appropriate vehicle, at concentrations known to be safe and effective, to combat superficial fungal infections (i.e., not systemic). Examples are athlete's foot plus oral and vaginal thrush. Methods of delivery may include, illustratively, stannous fluoride gel, cream, or similar suitable pharmaceutical preparations for the skin and mucous membrane, and a lozenge, pastille, or troche for the oral thrush and so on. The vaginal dosage form may be a suppository, cream, vaginal tablet, etc.

Various embodiments contemplate the use of stannous fluoride in an appropriate vehicle, at concentrations known to be safe and effective, to combat fungal infections of the lung. Fungal infections of the lung are not uncommon among asthmatics who use the corticosteroid-based inhalers for prevention of their respiratory disease (e.g., Azmacort®, Flovent®). The drug delivery device may comprise a Metered Dose Inhaler (MDI), a squeeze inhaler, a vaporizer, and the like.

### Eye Drop Embodiments

Various embodiments contemplate the use of stannous fluoride in an appropriate vehicle such as sterile eye drops, at concentrations known to be safe and effective, to combat herpes infections of the eye (Herpes simplex, Herpes zoster [Shingles] are not uncommon ophthalmic infections). Such a preparation may be used for treatment or prevention, especially if a Shingles or herpes simplex infection resides nearby. The consequences of untreated ocular herpes are severe. It is noted that Herpes zoster infections of the eye are most often caused by a Shingles outbreak on the forehead.

### Combination with Antimicrobial Agents

Various embodiments contemplate the use of stannous fluoride in an appropriate vehicle, at concentrations known to be safe, in combination with a suitable, known antimicrobial agent to broaden the spectrum of activity, i.e., antiviral, antifungal, and antibacterial.

In one embodiment, the antimicrobial agent may comprises GML or chlorine dioxide. In another embodiment, the antimicrobial agent may comprise a metallic element such as tin, silver, zinc or another metal. In one embodiment, any combination of any number of the antimicrobial agents described above may be used with SnF₂.

### Combination with GML

Various embodiments contemplate the use of stannous fluoride in an appropriate vehicle, at concentrations known to be safe and effective, in combination with glycerol monolaurate (GML), a commonly used food (and cosmetic) additive. GML is classed as a surfactant/preservative, though it is believed to also possess anti-viral activity. GML has been investigated to prevent toxic shock syndrome (TSS) caused by a bacterial strand known as Staphylococcus aureus and Streptococcus pyogenes, which is typically associated with the use of tampons. Various other embodiments are adapted to the therapeutic use and delivery of GML itself, such as utilizing its known antibacterial properties within the context of its inclusion in ice cream, deodorant, toothpaste, etc.

### Liquid Bandage Application

Various embodiments contemplate the use of stannous fluoride in a liquid bandage-like preparation that, when applied to the skin or mucous membrane, forms an antimicrobial barrier over a minor wound, thus guarding against infection. Examples may include 1) incorporating substances that migrate to the top of film, where they form chemical or physical bonds and/or 2) causing a drying effect only at the surface (i.e. interacting with air, much like paint does), but leaving the underlying stannous fluoride gel intact and/or 3) a separate material that provides the membrane and/or 4) a scrim which produces the membrane or uses other ingredients coated or impregnated into the scrim to form a protective surface coating.

### Hydrocolloid Dressing Application

Various embodiments contemplate a stannous fluoride-containing hydrocolloid dressing. The article is most appropriate for herpes infections of the skin and mucous membrane, like herpes simplex infections around the face, or herpes zoster outbreaks (Shingles) anywhere. If viscosity is an issue, it might be maintained in this way.

### Mucoadhesives

Various embodiments contemplate a stannous fluoride containing mucoadhesive dressing. Mucoadhesives are dosage forms that stick to mucous membranes and deliver drug to the area. Mucoadhesive dressings are most appropriate for herpes infections of the mucous membrane, such as for example, lips, mouth, genitalia eye area or an open wound.

Mucoadhesives are usually formulated from polymers, to which an active drug like stannous fluoride may be added. They form a protective film over a lesion and provide numerous advantages. One advantage is that the mucoadhesive may resist being rubbed off during everyday activities (e.g., wiping off the medicine on sheets and pillow cases while sleeping, eating, etc.). As a result, the number of daily applications that are required may be reduced. In addition, the mucoadhesive may allow the drug to maintain a necessary viscosity. Another advantage of using the mucoadhesive is that the drug may be applied in such a way to prevent the herpes virus from spreading. The mucoadhesive may also used to allow the drug to be delivered steadily over an extended period of time. In other words, the mucoadhesive provides a “sustained release” feature for topical dosage forms of SnF₂, much as sustained release qualities are incorporated into many extended release oral capsules and tablets. By keeping the gel in better and more prolonged contact with the affected area (e.g., the lips, the mouth, etc.) the efficacy is improved.

### Multiple Core Formulations and Dosages

The various embodiments discussed herein contemplate the use of different safe and effective doses and core formulations, such as a cream and other emulsions, or those that might offer unique attributes. In addition, various embodiments contemplate providing a means for delivering stannous fluoride gel 0.4% (or other concentrations) to the skin or mucous membrane. The means may be those that promise protection from being rubbed off, and convenience (currently the gel must be applied around six times daily, to keep the area permanently moist). Aside from cold sores, a novel platform formulation also may be applied, and have therapeutic benefit over other currently available gels sold widely for such indications as preventing dental caries and hypersensitive teeth.

### Additional Details Re Scrim Embodiments

Various embodiments contemplate the use of an absorbable scrim made out of a substance such as cotton or a synthetic fabric. When the scrim is immersed in the desired medicine or other therapeutic substance, it retains the substance(s) and holds it fast to the affected area of the skin or mucous membrane. The scrim may be applied to an area either external or internal to the body. Also, this scrim may be treated in a manner to hold the desired substance within the mesh such that it can be released at a later time, i.e., is delayed release.

The scrim can be coated or can absorb a medicine from being immersed in a bath. There are also various ways to modify the strength of the medicines' bond to the fabric, such as heat, pressure, mixing, etc. When this happens, the medicine is held by, or bonded to the scrim filaments. In order to release the medicine, a liquid which leaches the medicine out of the scrim is applied. When the medicine is released from the filaments of the scrim, it is incorporated into the leaching liquid, which contacts the desired area to be treated. This exposes the medicine to the affected area in varying concentrations on a timed-release schedule. The strength of the leaching agent, the method used to bond the medicine to the scrim filaments, the synthetic or natural filaments used, and the medicine(s) applied to the scrim shute and warp filaments creating the desired release concentrations of any number of medicines and how these medicines are released over time to the wound area. For example, one could release a medicine to first cleanse a wound, followed by a topical antibiotic, then a debriding agent, culminating in a protective covering of these otherwise undesirable materials on the surface, away from the wound. There are endless possibilities with these three variables, hundreds of natural and synthetic fibers with varying properties, many leaching agents, and a plethora of medicines or other therapeutic materials.

In various embodiments, the “Z” or third dimension of the scrim may increase the surface area of the scrim relative to the leaching liquid or semi-liquid by crimping the material or just making it thicker in the “Z” direction, thus, exposing much more surface area of the scrim to the liquid and this factor may also be used to design the time release properties of the scrim. The scrim may also be made of a polymer with specific bonding properties so to attract the medicine or desired substance and the design of the scrim along with the leaching liquid or semi-liquid may deliver a predetermined time-release schedule.

The valence of the bonding scrim alone or in conjunction with electrical or magnetic properties of the scrim design may also be used to hold and release a medicine or desired substance on a specific timetable. An external factor such as, for example, electricity, electromagnetic waves, microwaves, sound waves, ultra sound waves, heat/cold or other external factors, may be used to release the medicine or desired substance from the scrim in a controlled fashion for which the release control will be dictated by the application of this external factor. An external energy source can change the geometry of a crimped scrim by straightening or compression the scrim which directly will change the open area of the scrim. A pain relief medicine can be incorporated into the scrim and released in a time fashion or as needed by the treated patient through the addition of an external energy source. A crimped scrim can be put on the outside of an area which you want to open up or restrict as needed. This process can be used to help close wounds.

The scrim design, how it holds fast the medicine or desired substance and the release effects caused by the external source provide a unique way to deliver a medicine or desired substance on any time table desired, such as a nonlinear or batched timetable.

In various embodiments, the dosage is adjusted by taking a physical or chemical measurement as to how the dosage of the medicine or desired substance is achieving the results that are desired, i.e., plotting a dose-ranging curve. If release is too fast, the delivery may be slowed down and if the release is too slow the delivery may be sped up. This is useful on both external and internal body applications. For an internal application, the release may also be activated by the consumption of a liquid or other substance, possibly even a substance added to an intravenous bag that may activate the release mechanism.

### Additional Details Re Combination Embodiments

In various embodiments, GLM is added to stannous fluoride in a powder, liquid or gel form via normally accepted mixing or milling practices so to disperse the substance uniformly at the appropriate particle size. GML is a GRAS (generally regarded as safe) substance which has been used for almost a century as a preservative with known properties to reduce or retard bacteria growth. The antimicrobial properties of stannous fluoride would be added to those of GML to broaden the antimicrobial spectrum of the final preparation. GML is added to stannous fluoride at a percentage to maximize the desired effect of reducing bacterial growth and possible retard fungus and/or other viral growth as a combination product. This percentage addition is from 0.1% to 90.0% and may include the addition of another dilutive non- active or standard dilutive substance to achieve the desired viscosity, flow properties or other application considerations so to present the end product in the best use configuration. This combination formulation may be used in, for example, the following applications.

1. Applied to topical sores, openings in the skin or sites of viral, bacterial or fungal nature. Topical sores may be herpes cold sores of the mouth and lips, or the genital/rectal areas of the body. It may also be used on Shingles whether or not there is a topical open sore or other such afflictions, largely to prevent super infections. Accordingly such applications may reduce and/or eliminate the outbreak, shorten healing time and/or reduce the size of an outbreak. It may also reduce itching, pain, discomfort and a burning feeling by providing a soothing feeling over time.

2. Formulated in a spray or mist form to include an inhaler so to treat viral, bacterial or fungal problems in the lungs, nasal passages or anywhere in the respiratory system.

3. Applied to a wound dressing used either internal or external to the body.

4. Applied to, embedded in or bonded to a mesh used in a facial mask or in an air filtration system so to reduce, or minimize air borne virus, bacteria or fungus.

5. When applied to a substance, the substance may acquire imported a charge, valence or any bonding method so as to attract the undesirable airborne substances.

6. May be used for infections of the eyes being applied in normal methods of applying liquids to the eye or eye region.

7. Added to a scrim or covering made of natural or synthetic material so to provide a barrier to prevent bacteria, virus and mold spores from contacting an open wound or burn area while allowing the area to breath and heal normally without creating a scab which retards the healing process. This is a breathable product used to cover the affected area and establish a shield to prevent bacteria, virus and mold spores from contacting the wound. The open area of the mesh or scrim along with technology used in making fabrics such as ‘Gortex’ type process may apply.

8. The product may be applied as a gel or cream to provide the same desired results as listed in #7.

9. Used for any known medical condition where an individual viral, bacterial or fungal infection may expand to a condition with multiple infections (i.e., “opportunistic” or “super infections”).

10. By virtue of its expanded spectrum of antimicrobial activity, may be applied to any material or device used or implanted internally in the body to reduce or prevent the potential of a viral, bacterial or fungal occurrence.

11. May be applied to or incorporated in the natural or synthetic fiber manufacturing process via known technology to add a liquid substance so to attract, hold or disable airborne viruses, bacteria or fungi. This combination formulation may be applied to clothing or materials used in an operating, clean or any room in which the reduction of air borne contaminants is considered vital.

12. May be incorporated in a mask or clothing worn around the neck to attract air borne substances which may cause allergies with associated complications. The substance or materials to which it is applied may acquire a charge with which to attract the air borne particles with an opposite charge.

13. May be applied to incise drapes or other medical coverings used in the operating room to prevent infections.

14. Specifically may be incorporated in lozenge form, dissolvable wafer/thin sheet or as an inhaler to prevent or reduce the duration of a common cold, flu or other respiratory infections.

15. GML alone or added to stannous fluoride gel may be added to a liquid which is then used in a bath, spray or electronically charged process to bind the GML and/or SnF₂ to a natural or synthetic fabric. This fabric with bonded GML and/or SnF₂ will have a second substance applied to stannous fluoride or other medically used substances so to release the GML and/or SnF₂ in a time dependent relationship which may deliver the GML and/or SnF₂ progressively over time as desired. The GML and/or SnF₂ slurry may also be incorporated in a molded scrim with openings which may be filled by capillary action or other methods to achieve the same timed-release specifications.

16. In an inhaler, may be used prophylactically as a topical nasal or oral inhaler, or other misting application prior to the start of a viral, bacterial or fungal outbreak in order to reduce or prevent the outbreak.

17. The warp and shute filaments of a woven fabric may be independently treated by a substance such as GML, stannous fluoride or other active ingredients to provide specific desired and targeted delivery of the active substances over time, and whose time delivery may be adjusted by the treatment of the different fibers, whether natural or synthetic. The warp fibers in one direction may be pretreated prior to the weaving process with one substance and the shute fibers may be pretreated with a different substance also prior to the weaving process. The fibers may be chosen for specific properties such as how tightly they may hold and active ingredient and how slow or fast they may release the active ingredient. Fibers may also be chosen such that the release of the active ingredient is activated by an external source such as heat, a liquid, microwaves, sound waves, light waves, radio waves, ultra sound waves or other common industrial technologies which may impart energy into or on a substance to obtain a desired result. The application of such an external source may release a substance when the outside technology is applied or cause a substance to bond with another substance thus creating a new compound which provides unique properties to the scientific resolution of a cosmetic issue, disease or affliction. By enabling a release of a substance through the application of an external technology or substance may provide a unique delivery system which may be activated when needed. It may allow for testing to determine if the dosage of what is being delivery is sufficient to obtain the desired results and then allow the individual or care giver to modify the dosage and time delivery as deemed necessary. In other words, fine-tuned custom therapy.

18. The addition of GML to stannous fluoride may be enhanced by known natural substances which add to or increase the effectiveness of the combination when treating viral, bacterial or fungal infections.

19. The release of active ingredients or inactive ingredients from either the warp or shute filaments of a scrim can be activated in a sequential manner by either a leaching solution or and external energy source. One such sequence could first release ingredients which cleanse an area, then provide a treatment, then remove unwanted material and then provide a scab over effect possible using the unwanted materials as part of the protective membrane. This process can be time sensitive and include pain release materials in a planned release fashion or as needed by the end user.

### Additional Details Re Combination/Particle Embodiments

Various embodiments contemplate the use of stannous fluoride alone and in combination with GML or any other known or accepted synthetic or natural substance including any known antimicrobial agent to broaden its' spectrum of activity and/or to prevent or reduce the growth of viruses, bacteria or fungi by filters or other methods of attraction which will bind these microorganisms and bar them from reaching patients who may generally develop resulting conditions and disease if normally exposed.

Two main areas are discussed herein with respect to industrial or personal use, along with three methods to attract, hold and contain unwanted airborne particles. Specifically, as discussed above, stannous fluoride may be (1) applied to filter media and (2) applied to clothing, drapes or any other material which is used to attract and hold unwanted air borne particles. This by virtue of the compound being divalent, thus attracting other ionized particles.

Various mechanism may be employed to attract, hold and/or contain unwanted airborne particles, such as:

(1) Standard filter media which traps and collects particles by passing air containing the unwanted particles through filter media with fine enough open area so to allow air through but trap particles of the desired size.

(2) Filter media with an applied charge which is opposite to the charge which exist in the air borne particles thus attracting and holding those opposite charged air borne particles. The charge may be inherent as a result of the materials used or applied by an external technology such as corona discharge or ozone treatment.

(3) Viscous material(s) applied to the filter media which causes the unwanted particles to become stuck to and/or trapped in the viscous material.

Stannous fluoride alone and in combination with GML or any of the know or accepted synthetic or natural substance is added to a filter media and/or to any natural or synthetic fiber product whose use is to attract and hold air borne viruses, bacteria and fungal spores.

Various products may be added to or incorporated within the desired media via a bath operation, liquid spray application, powder spray operation, solvent coating operation, calendaring operation, paper making operation, carding operation or any other known or standard industrial operation used to make fabric, screens or filter media.

Normal filter media captures air borne particles by designing the open area of the filters to be smaller than the particle size it desires to stop. When one tries to stop very small particles, the air flow through the filters become drastically reduced and the filter size becomes very large to handle any reasonable volume of air. The applied substance may attract and hold the air borne particles by design, thus, allowing the open area of any filter to be larger which may enable greater flow and through put of air. This may enable the filtering system to be more efficient, lower cost, smaller and/or provided improved results. If a crimped warp and/or shute filament is used in the filter construction then the open area of the filter could be reduced or expanded when an external energy source is applied. As a filter loads up with material, the open area can be automatically adjusted to keep the open area constant automatically by measuring the pressure drop across the filter. This will improve operating efficiency, cost and maintenance. When the filter can no longer be opened up, it can be washed and returned to its original state and continuously used thus saving money on replacement filters and their disposal and improving operating efficiency.

Various embodiments accomplished this by one of the three methods listed above. Additionally, some embodiments use corona discharge and/or ozone treatment technologies for applying the charge to the media. The products may also be added to any textile operation which always has a liquid part of their process. Textile fibers either natural or synthetic are used in anything woven which covers an enormous range of products, such as face masks.

Face masks are often worn by people who have a cold and do not want to pass that cold to others. Such a face mask is also used to stop one from getting a cold. A face mask may also be used by individuals who get allergies from air borne particles. This may be a particular use for military personal deployed on submarines that are exposed to very refined and filtered air for months at a time. When those submarines resurface and those military personal depart, most get an upper respiratory infection due to the fact that they had not been exposed to any germs for an extended period of time. This may also apply to space travel.

A more universal application is on normal air travel, especially internationally. The air filtration systems are different for the passengers than the pilots. Travelers who have a disease that may easily become airborne may quickly pass their disease to other passengers. This happens frequently. Filter systems on airplanes are not designed to remove fine air borne particles such as viruses, bacteria or fungal spores. Additionally, different areas of the world are affected by different viruses, bacteria and fungi and this is why many international travelers acquire an upper respiratory infection several days after they travel. Our technology can be used within a plane's air filtration systems or alternatively the passengers may wear facial masks impregnated with the product(s).

If the attraction of the air borne particles is a result of the opposite charge on the substance that is applied to a fabric, then this feature may be applied to clothing. A person's clothing may then attract and hold the airborne particles, thus preventing those particles from being inhaled and causing a viral (cold), fungal, or other infection, or allergic reaction. A spray and/or pump system may be used to dispense the product(s) which may then apply the product(s) to one's clothing especially around the head and shoulder area so to attract air borne particles which are in close proximity to ones breathing space. One may also apply the substance(s) to small disposable inserts that are placed in the nostril, which may then attract and hold unwanted air borne substances. Those particles near the mouth area are then attracted and held by clothing worn around or in the neck area. This may not eliminate all unwanted airborne particles but may reduce their introduction into the body so to reduce or mitigate conditions or diseases caused by microorganisms and particles like pollen, dander or other airborne allergens.

### Combination with Wound Healing Agents

Various embodiments contemplate the use of stannous fluoride in an appropriate vehicle, at concentrations known to be safe and effective, in combination with one or more suitable topical wound healing agents, those that prevent the formation of a scab over a lesion that has begun to heal. The lesion may have been formed by trauma and/or infection. Ordinarily a scab begins to form after the lesion rids itself of the causative agent, usually a microbe. The wound healing agent ordinarily would retard the normal formation of a scab, thus providing cosmetic benefit. In sum, the stannous fluoride addresses the microbial infection, and the wound healing agent retards the scab, thus reducing the visible healing time of the lesion.

Scabbing is a normal physiologic healing process in man or other animals that enables broken skin or mucous membrane to repair itself. Ordinarily a scab appears as an unsightly blemish that may last from 5-10 days before it sloughs off naturally.

Sometimes referred to as “protectants”, topical wound healing agents are well known. They range from white petrolatum to Vitamin E oil, to honey. Here, they may be treated as either active or inactive ingredients (excipients, vehicles). As stannous fluoride may not be chemically and/or physically compatible with a particular wound healing agent, those skilled in the art would be able to formulate a cream, emulsion, or similar dosage form suitable for application to the skin or mucous membrane.

### Chlorine Dioxide and/or Chlorite

Any of the various embodiments of delivery methods discussed above may further include the use of chlorine dioxide (ClO₂) and/or also referred to as chlorite. Chlorite is an antimicrobial agent particularly effective against fungi.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

What is claimed is:

1. A method of treating a herpes virus, comprising:
providing a scrim comprising a plurality of openings, wherein a size each one of the plurality of openings is a function of a surface tension and a viscosity of stannous fluoride (SnF₂) that is applied;
adding an effective amount of SnF₂ to the scrim;
applying the scrim containing the effective amount of SnF₂ to an area of a body infected with the herpes virus.

2. The method of claim 1, wherein the effective amount of SnF₂ comprises approximately 0.4% up to 40%.

3. The method of claim 1, further comprising:
adding an antimicrobial agent to the scrim in combination with the effective amount of SnF₂.

4. The method of claim 3, wherein the antimicrobial comprises at least one of glycerol monolaurate (GML) or chlorine dioxide (ClO₂).

5. The method of claim 3, wherein the antimicrobial agent comprises a metallic element.

6. The method of claim 1, wherein the scrim comprises at least one of: a natural fiber or a synthetic fabric.

7. The method of claim 1, further comprising:
adding a liquid after applying the scrim to the area of the body infected with the herpes virus to provide a timed-release of the effective amount of SnF₂.

8. The method of claim 6, further comprising:
increasing a z-direction of the scrim to improve the timed-release.

9. The method of claim 7, wherein the increasing is achieved by crimping the scrim.

10. The method of claim 1, wherein the scrim is applied to an external area of the body or an internal area of the body.

11. A method of treating a herpes virus, comprising:
adding an effective amount of SnF₂ to a liquid or a semisolid;
applying the liquid containing the effective amount of SnF₂ to an area of a body infected with the herpes virus.

12. The method of claim 11, wherein the effective amount of SnF₂ comprises approximately 0.4% up to 40%.

13. The method of claim 11, further comprising:
adding an antimicrobial agent to the liquid in combination with the effective amount of SnF₂.

14. The method of claim 13, wherein the antimicrobial comprises at least one of glycerol monolaurate (GML) or chlorine dioxide (ClO₂).

15. The method of claim 13, wherein the antimicrobial agent comprises a metallic element.

16. The method of claim 11, wherein the applying comprises application of the effective amount of SnF₂ via at least one of: a nasal mist, a metered dose inhaler, a squeeze inhaler or a vaporizer.

17. The method of claim 11, wherein the liquid containing the effective amount of SnF₂ is applied via a liquid eye drop.

18. The method of claim 11, wherein the liquid containing the effective amount of SnF₂ is applied via a liquid bandage.

19. A method of treating a herpes virus, comprising:
adding an effective amount of SnF₂ to a mucoadhesive;
applying the mucoadhesive containing the effective amount of SnF₂ to an area of a body infected with the herpes virus.

20. A method of treating a herpes virus, comprising:
formulating an effective amount of SnF₂ into a lip balm;
forming the lip balm to accommodate a solid lip balm applicator;
applying the lip balm containing the effective amount of SnF₂ to an area of a body infected with the herpes virus using the solid lip balm applicator.
